(19) United States
(12) Patent Application Publication
(10) Pub. No.: US 2008/0114455 A1
(43) Pub. Date: May 15, 2008

(54) Rotating Interspinous Process Devices and Methods of Use

(75) Inventors: Eric C. Lange, Collierville, TN (US); Kidong Yu, Memphis, TN (US)

Correspondence Address: COATS & BENNETT/MEDTRONICS, 1400 CRESCENT GREEN, SUITE 300, CARY, NC, 27518, US

(73) Assignee: WARSAW ORTHOPEDIC, INC., Warsaw, IN (US)

(21) Appl. No.: 11/560,016

(22) Filed: Nov. 15, 2006

(51) Int. Cl. A61F 2/44

(52) U.S. Cl. 623/17.16

(57) ABSTRACT

The present application is directed to devices and methods for spacing vertebral members. The devices may include a body with major and minor axes. A first pair of opposing sides may be disposed along the minor axis and a second pair of opposing sides may be disposed along the major axis. The device may be rotated from a first orientation with the first pair of opposing sides facing towards the spinous processes to a second orientation with the second pair of opposing sides in contact with and spacing apart the spinous processes. Methods may include inserting a body within an interspinous space between the first and second spinous processes. The body may be inserted in a first orientation with a major axis of the body being aligned with a centerline of the space. After insertion, the body is rotated to a second orientation with the major axis being aligned across the centerline of the space and contacting opposing ends of the body against inner edges of the spinous processes and spacing apart the spinous processes.

### BACKGROUND

The present application is directed to devices and methods for spacing vertebral members, and more particularly, to interspinous devices positioned between the spinous processes for spacing the vertebral members.

Vertebral members comprise a body, pedicles, laminae, and processes. The body has an hourglass shape with a thinner middle section and wider sides, and includes sideplates on the inferior and superior sides. The pedicles are two short rounded members that extend posteriorly from the body, and the laminae are two flattened members that extend medially from the pedicles. The processes are projections that serve as insertion points for the ligaments and tendons. The processes include the articular processes, transverse processes, and the spinous process. The spinous process is a single member that extends posteriorly from the junction of the two lamina. The spinous process may act or be used as a lever to effect motion of the vertebral member. Intervertebral discs are positioned between the bodies of adjacent vertebral members to permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, loss of motion, loss of stability, and loss of disc height with or without impingement of neurologic tisue.

One method of correcting these conditions is insertion of a device between the spinous processes of adjacent vertebral members. The device may reduce or eliminate the pain and neurological deficit by increasing the disc height, adding stability, or restoring normal motion patterns.

### SUMMARY

The present application is directed to devices and methods for spacing vertebral members. The devices may include a body with major and minor axes. A first pair of opposing sides may be disposed along the minor axis and a second pair of opposing sides may be disposed along the major axis. The device may be rotated from a first orientation with the first pair of opposing sides facing towards the spinous processes to a second orientation with the second pair of opposing sides in contact with and spacing apart the spinous processes.

Various methods are disclosed for spacing apart the vertebral members. The methods may include inserting a body within an interspinous space between the first and second spinous processes. The body may be inserted in a first orientation with a major axis of the body being aligned with a centerline of the space. After insertion, the body is rotated to a second orientation with the major axis being aligned across the centerline of the space and contacting opposing ends of the body against inner edges of the spinous processes and spacing apart the spinous processes.

### BRIEF DESCRIPTION OF THE DRAWINGS

FIG. 1 is a perspective view of a device according to one embodiment.

FIGS. 2 is a side view of a device in a first orientation inserted between spinous processes according to one embodiment.

FIG. 3 is a side view of a device in a second orientation inserted between spinous processes according to one embodiment.

FIG. 4 is a perspective view of a device according to one embodiment.

FIG. 5 is a cross-sectional view taken along line V-V of FIG. 4 of a device according to one embodiment.

FIG. 6 is an exploded view of a device according to one embodiment.

FIG. 7 is a perspective view of a device according to one embodiment.

FIG. 8 is a perspective view of a device according to one embodiment.

FIG. 9 is a perspective view of a device according to one embodiment.

FIG. 10 is a perspective view of a device according to one embodiment.

FIG. 11 is a perspective view of a device according to one embodiment.

FIGS. 12A and 12B are schematic views of a device according to one embodiment.

FIG. 13 is a schematic view of a device according to one embodiment.

FIG. 14 is an exploded view of a device according to one embodiment.

### DETAILED DESCRIPTION

The present application is directed to devices and methods for spacing apart vertebral members. The devices may include a body with a major axis and a minor axis. The body may be inserted between the spinous processes in a first orientation with the major axis aligned with the space between the processes, and the minor axis aligned towards the processes. The body may then be rotated with the major axis aligned across the space between the processes. This rotation causes the body to space apart the spinous processes to support and/or stabilize the vertebral members.

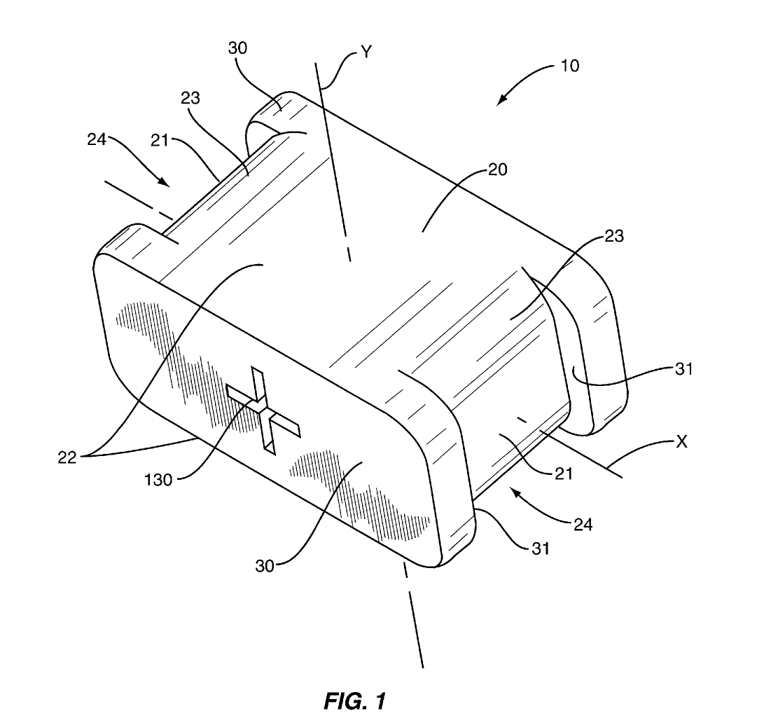

FIG. 1 illustrates one embodiment of a device 10. The device 10 includes a body 20 positioned between wings 30. The body 20 includes an elongated shape with a major axis X and a minor axis Y. The length of the body 20 in along the major axis X is larger than the length in the minor axis Y, with the amount of difference depending upon the context of use. Body 20 includes a first pair of sides 21 that opposing one another along the major axis X. Body 20 further includes an opposing second pair of sides 22 along the minor axis Y. Curved sections 23 may transition between the sides 21, 22.

The body 20 of FIG. 1 includes a substantially rectangular cross-sectional shape with sides 21 and 22 each being substantially planar and parallel. Other embodiments may include one or both side pairs being non-parallel and/or non-planar. FIG. 11 illustrates an embodiment with sides 22 including non-parallel and non-planar surfaces. In another embodiment, body 20 includes a substantially oval cross-sectional shape. A mount 130 may be positioned to receive a rotational tool to rotate the device 10. Mount 130 may be female or male, and may include a variety of shapes and sizes.

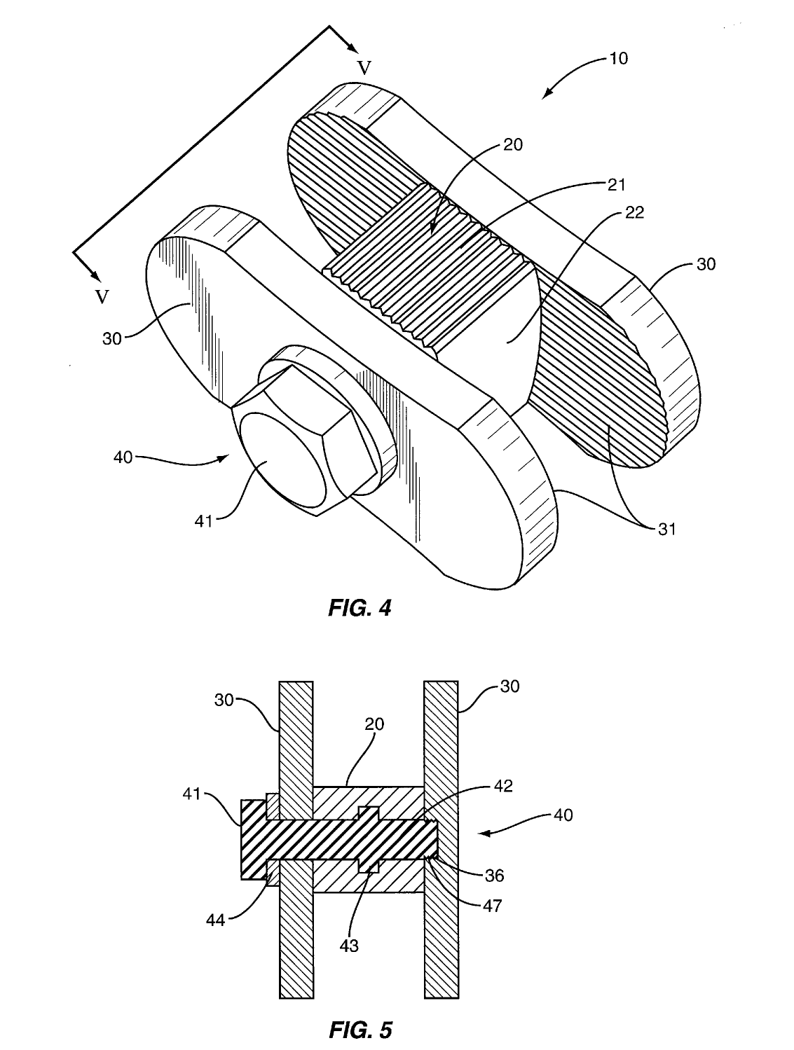

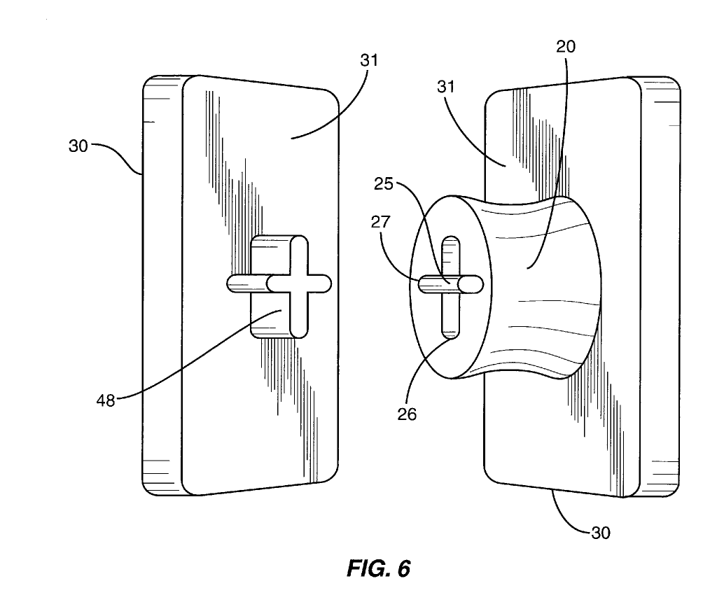

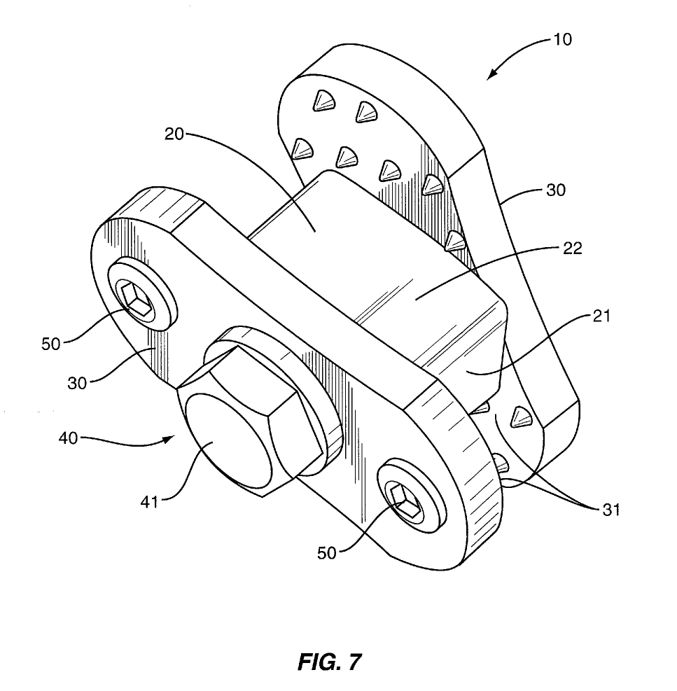

Sides 21 contact the spinous processes 91 when the device 10 is in a second orientation to space apart the vertebral members 90. Therefore, sides 21 may include a variety of shapes and surface configurations to maintain the contact. Sides 21 may be substantially smooth as illustrated in FIG. 1 to facilitate movement of the device 10 between first and second orientations. Sides 21 may also include serrations as illustrated in FIG. 4, or scallops as illustrated in FIGS. 6 and 7 to maintain the contact with the spinous processes.

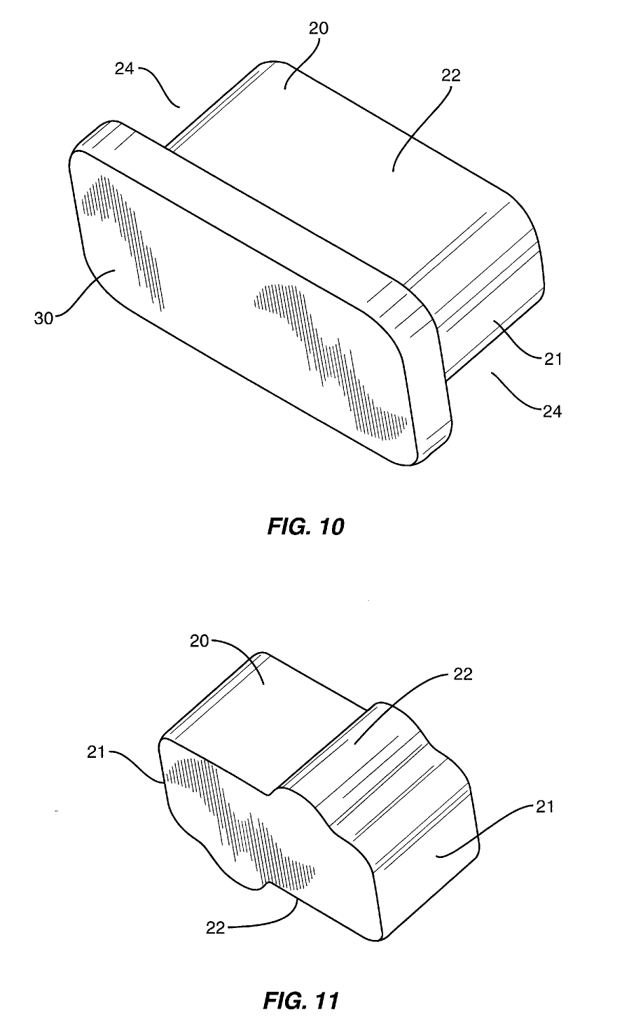

Wings 30 may extend from the body 20 for positioning the body 20 within the interspinous space. Embodiments may include a pair of wings 30 as illustrated in FIG. 1, a single wing 30 as illustrated in FIG. 10, or no wings as illustrated in FIG. 11. In multiple wing embodiments, the wings 30 may be substantially similar or they may include different shapes and/or sizes. The wings 30 may extend outward beyond the sides 21 to contact the spinous processes 91. A channel 24 may be formed at one or both of the sides 21 of the body 20. The channel 24 is sized to extend around a portion of the spinous process 91 when the body 20 is rotated to the second orientation. An inner surface 31 of the wings 30 may further include surface features to maintain the contact with the spinous processes 91. Surface features may include teeth and serrations. The surface features may extend across a limited area or the entirety of the inner surface 31. In multiple-wing embodiments, the surface features may be on both or just one of the wings 30.

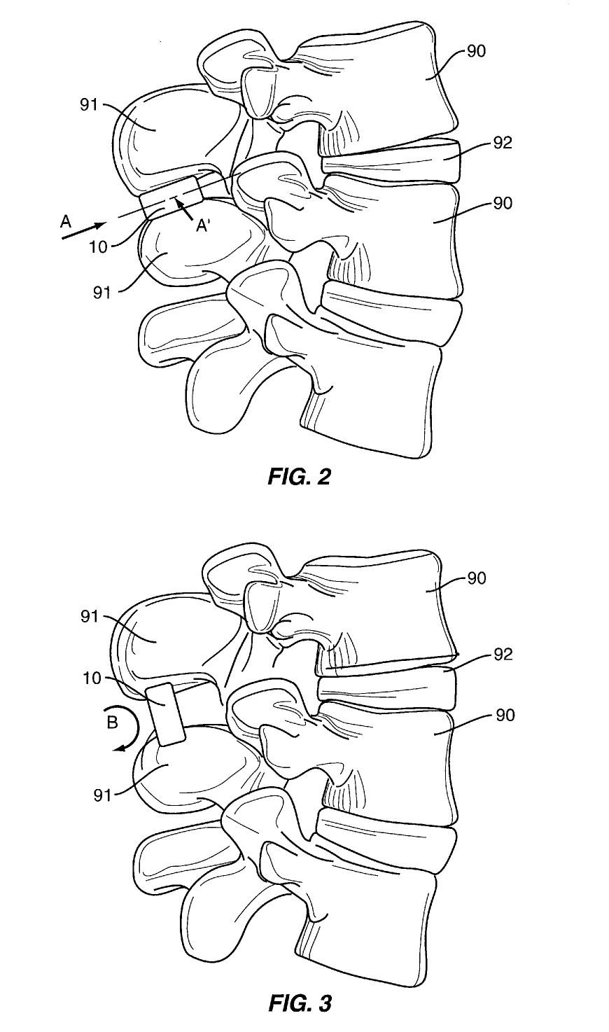

FIGS. 2 and 3 illustrate one embodiment of using the device 10. As illustrated in FIG. 2, the device 10 is inserted from either the posterior or lateral direction as indicated by arrows A and A′ into the space formed between the spinous processes 91. The device 10 is inserted with the major axis X aligned with the interspinous space and the sides 22 facing towards the processes 91. The device 10 may include a height along the minor axis Y such that the sides 22 are spaced away from the processes 91. Once inserted, the device 10 is rotated as illustrated by arrow B. The rotation causes the sides 21 to contact the spinous processes 91. The larger length of the major axis X spaces apart the spinous processes 91. This design is advantageous as there is no need for a separate distractor as the device 10 performs both distraction and spacing of the spinous processes 91.

The amount of rotation of the device 10 between the first and second orientations may vary. In the embodiment of FIGS. 2 and 3, a rotation of about 90° occurs between the first and second orientations. In other embodiments, the major and minor axes X, Y may not be perpendicular. Therefore, different amounts of rotation are necessary for the spacing.

The device 10 may be constructed as a unitary piece. FIG. 1 illustrates one example with body 20 and wings 30 constructed from a single piece. The construction of the device 10 may begin with a substantially rectangular member and forming channels 24 at one or both sides 21.

The device 10 may also be constructed of multiple parts that are attached together. By way of example, body 20 may be a separate member that is attached to the wings 30. This construction may allow the body 20 to rotate independently of the wings 30. A number of different structures may be used to attach the body 20 to the wings 30. FIGS. 4 and 5 illustrate one embodiment with a connector 40 that connects the body 20 to the wings 30. Connector 40 includes a head 41 and a shaft 42. The head 41 extends outward from one of the wings 30 and may be shaped to receive a tool. Rotation of the head 41 causes the shaft 42 and the body 20 to rotate relative wings 30. A washer 44 may be mounted between the head 41 and the wing 30. One or more extensions 43 may extend outward from the shaft 42 and into the body 20. The extensions 43 provide for the rotation of the shaft 42 to be transferred to the body 20.

Another attachment design is illustrated in FIG. 6. An extension 48 extends outward from the inner surface 31 of wing 30. Extension 48 includes an elongated shape that is sized to fit within an aperture 25 in the body 20. The aperture 25 includes a cross-cut or substantially “+” shaped configuration with first and second sections 26, 27. The sections 26, 27 are shaped and sized to receive the extension 48 and maintain the position of the body 20 relative to the wing 30. Sections 26, 27 also provide for rotation of the body 20 relative to the wings between the first and second orientations. Extension 48 fits within sections 26 in a first orientation, and within sections 27 in the second, rotated orientation. In use, the device 10 is inserted with the body 20 in a first orientation with the extension 48 within sections 26. After insertion, body 20 is rotated to the second orientation with the extension 48 moved to within sections 27. In one embodiment, one or both of the body 20 and extension 48 are deformable to allow for moving the extension 48 between sections 26, 27. The opposing wing of the FIG. 6 embodiment may also include an extension that fits within a corresponding aperture in the body 20. Opposing wing may also include a shaft that connects to the body 20 to provide for the body 20 to rotate.

FIG. 14 illustrates another embodiment with an extension 48 sized to fit within a corresponding aperture 25. Once inserted, the wing 30 is rotated with a head of the extension extending beyond the aperture 25 to lock to the wing 30 to the body 20. In another embodiment, extension 48 is inserted into the aperture 25 and the extension 48 is rotated to lock the members together.

In some multi-wing embodiments, the wings 30 are substantially parallel. In other embodiments, wings 30 may be non-parallel. Further, wings 30 may be substantially aligned as illustrated in FIGS. 1 and 4 with each of the wings 30 is positioned at substantially the same rotational position relative to the body 20. Wings 30 may also be aligned at different positions. FIG. 7 includes an embodiment with the wings 30 at different rotational positions relative to the body 20. Wings 30 may be at fixed rotational positions, or may be movable to adjust the position. The rotational position of the wing or wings 30 is set to contact the spinous processes 91 and maintain the position of the body 20 within the interspinous space. Apertures 50 within one or more of the wings 30 may be positioned to receive a fastener for attachment to the vertebral members.

Device 10 may further include a locking mechanism for maintaining the rotational position of the body 20 relative to the wing or wings 30. In one embodiment as illustrated in FIG. 5, the shaft 42 includes threads 47 that engage a threaded aperture 36 in the wing 30. Rotation of the shaft 42 through engagement of a tool with the head 41 causes the threads 47 to engage with the threaded aperture 36. The rotation causes the body 20 to be compressed between the wings 30 to maintain the rotational position. Another locking mechanism is included with the embodiment of FIG. 6. The shape of the extension 48 and the aperture 25 prevents the inadvertent rotation of the body 20 relative to the wings 30.

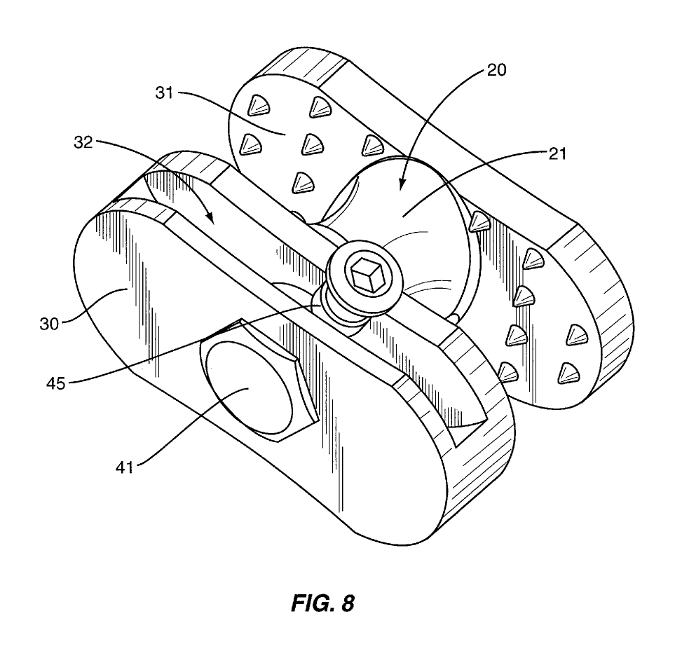

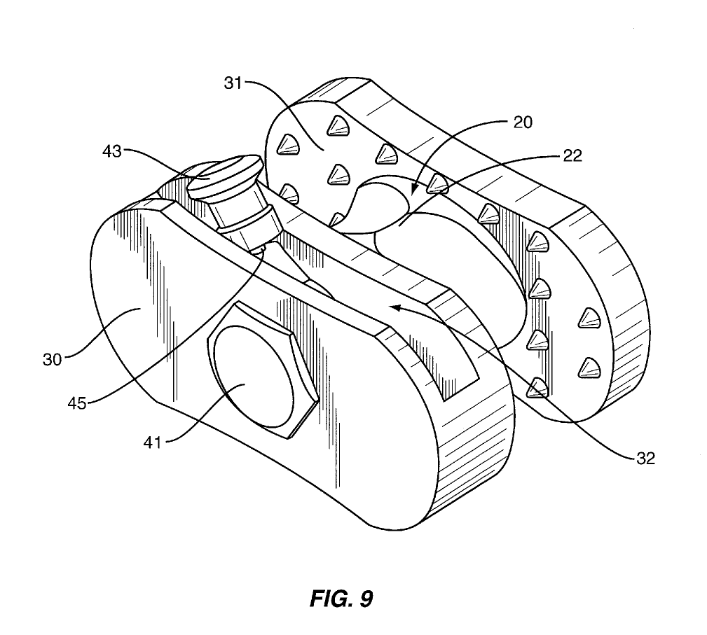

FIGS. 8 and 9 illustrate an embodiment for rotating the body 20. A lever 45 is connected to and extends outward from the shaft 42. An engagement surface, such as a polygonal indent, may be positioned at the end of the lever to receive a rotation tool to move the lever 45 and body 20. Pivoting movement of the lever 45 causes the body 20 to move between the first orientation as illustrated in FIG. 8 and the second orientation as illustrated in FIG. 9. Lever 45 may be sized to fit within a slot 32 formed within the wing 30. Lever 45 may also be positioned on the exterior of the wing 30 (i.e., away from the body), or on the interior of the wing 30 adjacent to the inner surface 31.

A locking mechanism may also lock the body 20 in the desired rotational position. In one embodiment, lever 45 may be threaded to engage with and lock to the shaft 42 that extends through the body 20. In another embodiment, lever 45 engages with the head 41 to lock the position. In yet another embodiment, the lever 45 does not contact the shaft 42 and rotation of the shaft 42 locks rotational position of the body 20. In the various embodiments, the lever 45 may fit within the slot 32, or be positioned away from the slot 32.

In one embodiment, device includes a body 20 without wings 30. FIG. 11 illustrates an example with the body 20 sized to space apart the vertebral members. Body 20 includes first sides 21 and second seconds 22.

Body 20 may be constructed of a rigid material that is able to support and space apart and the spinous processes 91. Materials may include metals, polymers, ceramics, and combinations thereof. Examples of metals include titanium, titanium alloys such as nickel-titanium, stainless steel, and cobalt chromium. Examples of polymers include silicone, silicone-polyurethane copolymer, polyolefin rubber, PEEK, PEEK-carbon composites, polyimide, polyetherimide, polyurethane, and combinations thereof. Examples of polyurethanes include thermoplastic polyurethanes, aliphatic polyurethanes, segmented polyurethanes, hydrophilic polyurethanes, polyether-urethane, polycarbonate-urethane, silicone polyetherurethane, polyvinyl alcohol hydrogel, polyacrylamide hydrogel, and polyacrylic hydrogel. Examples of ceramics include calcium phosphate, hydroxyapatite, HAPCP, alumina, and zirconium.

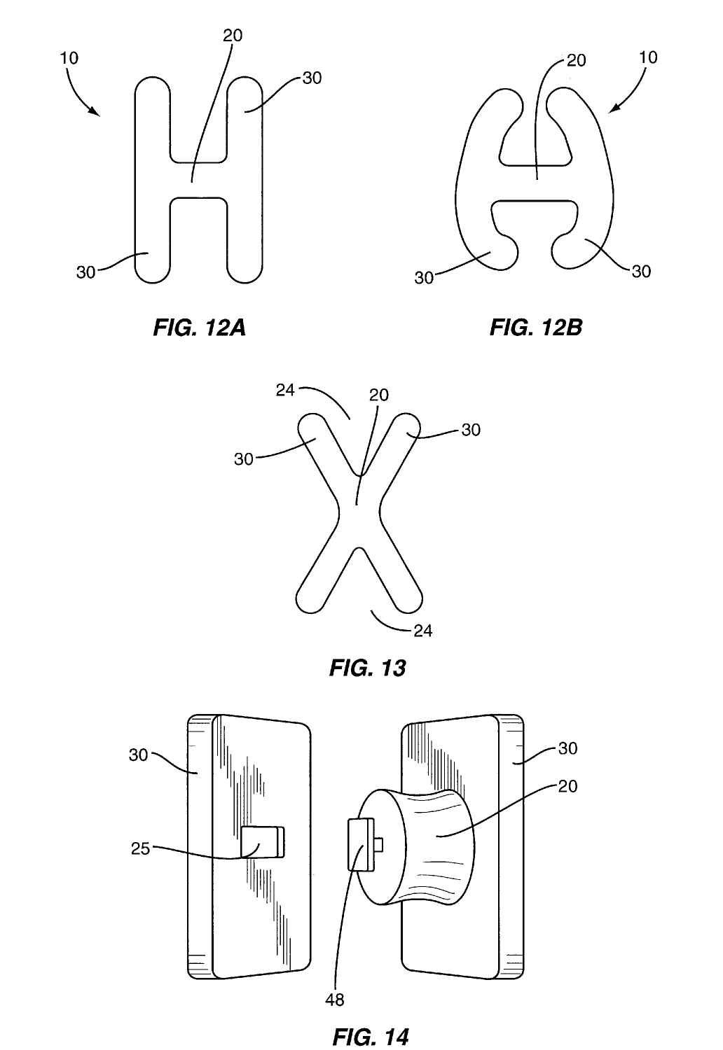

Body 20 and/or wings 30 may be constructed of a shape memory material as illustrated in FIGS. 12A and 12B. The device 10 includes a first shape as illustrated in FIG. 12A when initially inserted into the patient. The device 10 transitions to a second shape as illustrated in FIG. 12B at some time after insertion. In one embodiment, the body 20 and/or wings 30 are constructed of Nitinol.

In one embodiment as illustrated in FIG. 4, the body 20 without the wings 30 is initially inserted into the patient in a first rotational position. After insertion, body 20 is rotated to a second rotational position to space apart the spinous processes 91 as necessary. After rotation, one or more wings 30 are attached to the body 20 to maintain it at the second rotational position. In one embodiment as illustrated in FIG. 4, body 20 includes an opening sized to receive the connector 40. In another embodiment, fasteners are mounted through the wings 30 and attached to the spinous processes 91 to maintain the rotational position.

The device 10 may include a variety of shapes. FIG. 13 illustrates an embodiment with the wings 30 extending outward at an angle from the body 20. Channels 24 formed at the ends of the body 20 are substantially V-shaped

The device 10 may be used at various regions of the spine, including the cervical, thoracic, lumbar and/or sacral portions of the spine. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. In one embodiment, the cross-sectional shape of body 20 is oval. The present embodiments are, therefore, to be considered in all respects as illustrative and not restrictive, and all changes coming within the meaning and equivalency range of the appended claims are intended to be embraced therein.

What is claimed is:

1. A device to fit within an interspinous space formed between first and second spinous processes, the device comprising:
a rigid body to insert between the spinous processes with major and minor axes, the body including a first pair of opposing sides disposed along the minor axis and a second pair of opposing sides disposed along the major axis, the body further including curved surfaces extending between the first pair of opposing sides and the second pair of opposing sides to enable rotation; and
a pair of opposing wings that extend outward beyond the second pair of sides and form channels to receive the spinous processes;
the device being rotatable from a first orientation with the first pair of opposing sides facing towards the spinous processes to a second orientation with the channels receiving the spinous process and the second pair of opposing sides in contact with and spacing apart the spinous processes and the wings extending along the spinous processes.

2. The device of claim 1, wherein the body is rotatably mounted to the pair of wings.

3. The device of claim 1, wherein the body and the pair of wings are constructed as a single member.

4. The device of claim 1, wherein the second pair of opposing sides are substantially parallel and the first pair of opposing sides are substantially parallel.

5. The device of claim 1, wherein the first pair of opposing sides and the curved surfaces are smoother than the second pair of opposing sides to facilitate rotation from the first orientation to the second orientation and for the second pair of opposing sides to maintain contact with the spinous processes in the second orientation.

6. The device of claim 1, further comprising a shaft that extends through the body and into each of the pair of opposing wings to enable the body to rotate relative to the pair of wings, the shaft further including an enlarged head positioned at an end of the shaft to contact an exterior of one of the wings.

7. The device of claim 6, wherein the shaft includes extensions that extend outward from a centerline of the shaft and into the body such that rotation of the shaft causes the body to rotate from the first orientation to the second orientation.

8. The device of claim 1, further comprising a locking mechanism to lock the body at the second orientation.

9. The device of claim 1, further comprising a shaft that extends through an interior of the body to enable the body to rotate relative to the pair of wings, wherein a lever is operatively connected to the shaft and extends outward from the shaft to rotate the body between the first and second orientations.

10. A device to fit within an interspinous space formed between first and second spinous processes, the device comprising:
an elongated body with a first height in a first dimension measured between a first pair of sides, and a second larger height in a second dimension measured between a second pair of sides;
a pair of wings positioned along opposing edges of the body and extending outward beyond each of the second pair of sides;
a pair of channels formed at the second pair of sides;
the body being positionable within the interspinous space between a first rotational orientation with the first height being less than the interspinous space and the first pair of sides facing towards but being spaced away from the spinous processes, and a second rotational position with the second height being larger than the interspinous space with the second pair of sides contacting against an inner surface of the spinous processes and the wings contacting against outer surfaces of the spinous processes.

11. The device of claim 10, further comprising a shaft that extends through an interior of the body to connect the body to the pair of wings.

12. The device of claim 11, further comprising a lever that extends outward from the shaft and is movable between first and second positions to rotate the body between the first and second orientations.

13. The device of claim 12, wherein the lever is positioned within a slot formed within one of the pair of wings.

14. The device of claim 10, wherein body and the pair of wings are constructed as a single member.

15. The device of claim 10, wherein the first pair of sides is smoother than the second pair of sides to facilitate rotation from the first orientation to the second orientation, and the second pair of sides is rougher than the first pair of sides to maintain contact with the spinous processes in the second orientation.

16. The device of claim 10, wherein one of the wings includes an extension that extends outward from an inner side to mount within an aperture in the body.

17. A device to fit within an interspinous space formed between first and second spinous processes, the device comprising:
a body to insert between the spinous processes with major and minor axes, the body including a first pair of opposing sides disposed along the minor axis and a second pair of opposing sides disposed along the major axis;
a pair of opposing wings that extend outward beyond the second pair of sides and form channels to receive the spinous processes;
a shaft that extends through the body to attach the body to the pair of wings, the body being rotatable about the shaft from a first orientation with the first pair of opposing sides facing towards the spinous processes to a second orientation with the second pair of opposing sides in contact with the spinous processes; and
a locking mechanism adapted to lock the body in the second orientation.

18. The device of claim 17, wherein the shaft is threaded to engage with a threaded aperture within one of the pair of wings.

19. The device of claim 17, further comprising a lever that is connected to and extends outward from the shaft, the lever is movable between a first position to place the body in the first orientation and a second position to place the body in the second orientation.

20. The device of claim 19, further comprising a slot positioned within one of the pair of wings, the slot being sized to receive the lever.

21. The device of claim 17, wherein the body is constructed of a rigid material.

22. A method of spacing first and second spinous processes comprising the steps of:
inserting a body within an interspinous space between the first and second spinous processes, the body being inserted in a first orientation with a major axis of the body being aligned with the interspinous space;
rotating the body to a second orientation with the major axis being aligned across the interspinous space and contacting opposing ends of the body against inner edges of the spinous processes and spacing apart the spinous processes;
aligning wings positioned on lateral sides of the body against outer edges of the spinous processes to maintain the opposing ends of the body against the inner edges of the spinous processes; and
locking the body in the second orientation.

23. The method of claim 22, wherein the step of rotating the body to the second orientation comprises moving a lever operative attached to a shaft that extends through the body from a first position to a second position.

24. The method of claim 23, further comprising moving the lever within a slot formed within one of the wings.

25. The method of claim 22, further comprising engaging protrusions on an inner side of the wings with the outer edges of the spinous processes before rotating the body to the second orientation.

26. The method of claim 22, further comprising attaching the wings to first and second spinous processes after the step of rotating the body to the second orientation.

27. A method of spacing first and second spinous processes comprising the steps of:
inserting an elongated body within an interspinous space between the first and second spinous processes, the body being inserted in a first orientation;
rotating the body from a first orientation and sliding relatively smooth surfaces of a first pair of opposing sides disposed along a minor axis against the spinous processes; and
rotating the body to a second orientation and contacting relatively rough surfaces of a second pair of opposing sides disposed along a major axis against the spinous processes.

28. The method of claim 27, further comprising positioning wings that extend along the lateral sides of the body along outer edges of the spinous processes and maintaining the second pair of opposing sides against the spinous processes.

29. The method of claim 27, further comprising locking the body in the second orientation.

30. The method of claim 27, further comprising attaching the body to the first and second spinous processes after the step of rotating the body to the second orientation.
